(19) United States
(12) Patent Application Publication
(10) Pub. No.: US 2010/0305392 A1
(43) Pub. Date: Dec. 2, 2010

(54) THORACIC AORTA AND VAGUS NERVE STIMULATION

(75) Inventors: Yossi Gross, Moshav Mazor (IL); Amir Dagan, Kibbutz Megiddo (IL); Yotam Reisner, Kiryat Tivon (IL); Offer Glasberg, Zichron Ya'akov (IL); Nitai Hanani, Haifa (IL); Gal Ariav, Givaat Ada (IL)

Correspondence Address: EDWARDS ANGELL PALMER & DODGE LLP, P.O. BOX 55874, BOSTON, MA, 02205, US

(73) Assignee: Enopace Biomedical Ltd., Caesarea (IL)

(21) Appl. No.: 12/792,227

(22) Filed: Jun. 2, 2010

Related U.S. Application Data
(63) Continuation-in-part of application No. PCT/IL2009/000117, filed on Jan. 29, 2009.
Application No. PCT/IL2009/000117 is a continuation-in-part of application No. 12/023,896, filed on Jan. 31, 2008.
(60) Provisional application No. 61/183,319, filed on Jun. 2, 2009.
(60) Provisional application No. 61/331,453, filed on May 5, 2010.

(51) Int. Cl. A61M 1/10; A61N 1/36

(52) U.S. Cl. 600/17; 607/44; 607/39

(57) ABSTRACT

Apparatus and methods are described including identifying a subject as suffering from a condition selected from the group consisting of congestive heart failure, diastolic heart failure, and hypertension. In response to the identifying, an electrode is placed on a vagus nerve of the subject at a vagal site that is between (a) a vagal bifurcation with a thoracic cardiac branch of the subject, and (b) thoracic vagal branching into the esophageal plexus of the subject. The subject is treated by driving a current into the vagal site, via the electrode. Other embodiments are also described.

### CROSS-REFERENCES TO RELATED APPLICATIONS

The present application is a continuation-in part of PCT Application PCT/IL2009/000117 to Gross, filed Jan. 29, 2009, entitled “Intra-aortic electric counterpulsation,” which claims the benefit of and is a continuation-in-part of U.S. patent application Ser. No. 12/023,896 to Gross, filed Jan. 31, 2008, entitled “Intra-aortic electric counterpulsation.”

The present application claims the benefit of (a) U.S. Provisional Patent Application 61/183,319 to Reisner, filed Jun. 2, 2009, entitled “Thoracic aorta and vagus nerve stimulation,” and (b) U.S. Provisional Patent Application 61/331,453 to Dagan, filed May 5, 2010, entitled “Thoracic aorta and vagus nerve stimulation.”

All of the above-referenced applications are incorporated herein by reference.

### FIELD OF EMBODIMENTS OF THE INVENTION

Some applications of the present invention generally relate to implanted medical apparatus. Specifically, some applications of the present invention relate to apparatus and methods for treating congestive heart failure, diastolic heart failure, hypertension, and/or other conditions.

### BACKGROUND

Heart failure is a condition in which a problem with the structure or function of the heart impairs its ability to supply sufficient blood flow to meet the body's needs. The condition impairs quality of life and is a leading cause of hospitalizations and mortality in the western world. Treatment of heart failure is typically aimed at removal of precipitating causes, prevention of deterioration in cardiac function, and control of congestive state.

Hypertension, or chronic high blood pressure, is an extremely prevalent medical condition, which can lead to strokes, heart attacks, and heart failure. There are a variety of treatments that are available for treating hypertension, including lifestyle changes, and medication.

U.S. Pat. No. 7,167,751 to Whitehurst describes a method of using a small implantable stimulator(s) with at least two electrodes small enough to have the electrodes located adjacent to the vagus nerve. The small stimulator is described as providing a means of stimulating the vagus nerve when desired, and as being capable of being implanted via a minimal surgical procedure.

PCT Publication WO 09/095,920 to Gross describes apparatus including a sensing electrode configured to be implanted at a non-cardiac site in a vicinity of an aorta of a subject and to detect an electrical parameter of the aorta, and a control unit configured to receive the detected parameter and to generate an output in response to the detected parameter.

PCT Publication WO 07/013,065 to Gross describes apparatus including a bifurcation stent comprising one or more electrodes, the stent configured to be placed in a primary passage and a secondary passage of a blood vessel, and a control unit, configured to drive the electrodes to apply a signal to a wall of the blood vessel, and to configure the signal to increase nitric oxide secretion by the wall. Other embodiments are also described.

PCT Publication WO 07/113,818 to Cahan describes an implantable artificial pacemaker (AAP) comprising oscillatory means providing pulsating signals at a pre-selected frequency, wherein the pacemaker is pacing the aorta. The application also describes an AAP further comprising: (i) a plurality of sensors disposed internally or externally to the aorta; and (ii) a pacing means in which the AAP stimulates the aortic media, augmenting physiological aortic elastic recoil. An AAP further comprising a processor capable of obtaining information from sensors and triggering an appropriate contraction wave in the aortic media is also described. A method of managing aortic rhythm is also described, comprising: (i) implanting an AAP comprising oscillatory means; and (ii) pulsating signals at a preselected frequency, so as to pace the aorta and in which the pulsating signals are provided by synchronizing and coordinating activation impulses in a portion of the aorta using electrical impulses.

US Patent Application Publication 2007/0150009 to Kveen describes apparatus, systems, and methods that include a pacing apparatus having a stent electrode through which pulses of electrical current can be delivered. Stent electrodes are described as receiving energy for generating the electrical current from a variety of sources. Sources include one or more induction coils that can form at least a portion of the stent. Sources also include an implantable pulse generator coupled to a lead through which pulses of the electrical current are supplied to the stent electrodes.

U.S. Pat. No. 6,865,416 to Dev describes methods for inducing or increasing the vasodilation of a vessel. The patent further provides methods for inducing or increasing the flow of fluid through a vessel. An electrical impulse is applied to the vessel in order to induce or increase vessel vasodilation or to induce or increase the flow of fluid through the vessel.

US Patent Application Publication 2004/0106954 to Whitehurst describes a treatment of congestive heart failure (CHF) that includes implantation of the discharge portion(s) of a catheter and, optionally, electrode(s) on a lead, near the tissue(s) to be stimulated. Stimulation pulses, i.e., drug infusion pulses and optional electrical pulses, are supplied by a stimulator implanted remotely, and through the catheter or lead, which is tunneled subcutaneously between the stimulator and stimulation site. Stimulation sites include the coronary arteries, the aorta, the left ventricle, the left atrium, and/or the pulmonary veins, among other locations. Disclosed treatments include drugs used for acute treatment of CHF, for chronic treatment of CHF, and drugs to reverse CHF.

US Patent Application Publication 2004/0054384 to Nachum describes a treatment method and device for promoting a localized increase in the flow of blood through a blood vessel in an area of the body, the method including the steps of: (a) providing a system including: (i) at least a first electrode operatively contacting a first portion of body tissue; (ii) at least a second electrode operatively contacting a second portion of body tissue; and (iii) a signal generator, operatively connected to the first electrode and the second electrode, for providing a plurality of electrical impulses to the electrodes; (b) applying the electrical impulses so as to subject the muscular tissue to at least one voltage differential, thereby inducing repeated, contracting, directional movement of muscular tissue associated within the blood vessel, so as to produce a localized increase in the flow of blood through the blood vessel.

U.S. Pat. No. 5,372,573 to Habib describes a method and apparatus for improving the flow of blood through a region of increased impedance. The method comprises assisting blood flow in said region by means of a pump placed in or around a blood vessel supplying blood to said region, and acting to pump blood in the required direction. The pump comprises, in one application, housing annularly surrounding a compressible conduit, said housing containing a plurality of flexible inflatable containers mounted for contact with said conduit (e.g., a blood vessel) and means for effecting sequential inflation and deflation of said containers so as to create a peristaltic pumping effect.

Biosense Webster, Inc. (CA, USA) manufactures the LASSO 2515 Variable Circular Mapping Catheter.

Sulzer IntraTherapeutics Inc. manufactures the IntraCoil® Self-Expanding Peripheral Stent (IntraCoil® Stent), which is described as a flexible coil-shaped metallic device that is used in the femoral and popliteal arteries in the leg to hold open areas that were blocked by atherosclerotic disease.

CardioMEMS, Inc., manufactures the EndoSure® Wireless AAA Pressure Measurement System, which is composed of two components: a miniaturized, wireless implantable sensor and an external electronics module. The external electronics module is described as wirelessly communicating with the sensors to deliver patient data. The wireless sensors are described as being powered by RF energy transmitted from an external electronics module and transmitting real-time data without batteries.

Cheetah Medical Inc. manufactures the Cheetah Reliant, which is described as providing continuous tracking of cardiac output and other parameters of cardiac function such as ventricular ejection time and heart rate.

The following references may be of interest:

U.S. Pat. No. 5,324,323 to Bui

U.S. Pat. No. 5,669,924 to Shaknovich

U.S. Pat. No. 5,782,774 to Shmulewitz

U.S. Pat. No. 5,900,433 to Igo

U.S. Pat. No. 5,904,712 to Axelgaard

U.S. Pat. No. 5,906,641 to Thompson

U.S. Pat. No. 5,913,876 to Taylor

U.S. Pat. No. 5,935,077 to Ogle

U.S. Pat. No. 6,038,485 to Axelgaard

U.S. Pat. No. 6,058,331 to King

U.S. Pat. No. 6,086,527 to Talpade

U.S. Pat. No. 6,106,477 to Miesel

U.S. Pat. No. 6,200,259 to March

U.S. Pat. No. 6,245,103 to Stinson

U.S. Pat. No. 6,347,247 to Dev

U.S. Pat. No. 6,463,323 to Conrad-Vlasak

U.S. Pat. No. 6,485,524 to Strecker

U.S. Pat. No. 6,721,603 to Zabara

U.S. Pat. No. 6,810,286 to Donovan

U.S. Pat. No. 6,824,561 to Soykan

U.S. Pat. No. 6,845,267 to Harrison

U.S. Pat. No. 6,871,092 to Piccone

U.S. Pat. No. 6,939,345 to Ken Knight

U.S. Pat. No. 7,082,336 to Ransbury

U.S. Pat. No. 7,090,648 to Sackner

U.S. Pat. No. 7,206,637 to Salo

U.S. Pat. No. 7,229,403 to Schock

U.S. Pat. No. 7,269,457 to Shafer

US 2002/0103454 to Sackner

US 2003/0036773 to Whitehurst

US 2003/0204206 to Padua

US 2004/0039417 to Soykan

US 2006/0229677 to Moffit

US 2006/0217772 to Libbus

US 2006/0276844 to Alon

US 2007/0196428 to Glauser

US 2007/0248676 to Stamler

US 2008/0058872 to Brockway

US 2009/0062874 to Tracey

PCT Publication WO 00/002501 to Benjamin

PCT Publication WO 04/014456 to Allen

PCT Publication WO 06/094273 to White

PCT Publication WO 06/064503 to Belsky

PCT Publication WO 06/123346 to Alon

PCT Publication WO 07/064,895 to Meyerhoff

PCT Publication WO 07/106,533 to Stern

PCT Publication WO 07/113,833 to Cahan

PCT Publication WO 08-100390 to Walker

PCT Publication WO 09/095,918 to Gross

European Patent Application Publication EP 0 109 935 A1 to Charmillot

“Vagus nerve stimulation as a method to temporarily slow or arrest the heart,” by Matheny, Ann Thorac Surg. 1997 June; 63(6 Suppl):S28-9

“Vagus nerve stimulation decreases left ventricular contractility in vivo in the human and pig heart,” by Lewis, J. Physiol. 2001 July 15; 534 (Pt 2): 547-552

“Sympathovagal balance is major determinant of short-term blood pressure variability in healthy subjects,” by Laitinen, Am J Physiol Heart Circ Physiol 276:1245-1252, 1999

“Preparation and characterization of implantable sensors with nitric oxide release coatings,” by MC Frost, Microchemical Journal Vol:74 Issue: 3, June, 2003 pp: 277-288

“Optimal frequency ranges for extracting information on cardiovascular autonomic control from the blood pressure and pulse interval spectrograms in mice,” by Baudrie, Am J Physiol Regul Integr Comp Physiol 292: R904-R912, 2007

“Neural influences on cardiovascular variability: possibilities and pitfalls,” by Malpas, Am J Physiol Heart Circ Physiol 282: H6-H20, 2002

“Improving the thromboresistivity of chemical sensors via nitric oxide release: fabrication and in vivo evaluation of NO-releasing oxygen-sensing catheters,” by MH Schoenfisch, Anal. Chem., 72 (6), 1119-1126, 2000

“Improving the biocompatibility of in vivo sensors via nitric oxide release,” by Jae Ho Shin, Analyst, 2006, 131, 609-615

“Heart rate variability,” by Task Force of the European Society of Cardiology and the North American Society of Pacing and Electrophysiology, European Heart Journal (1996) 17, 354-381

“Heart rate and vasomotor control during exercise,” by Vallais, Proceedings of the 29th Annual International Conference of the IEEE EMBS, Cite Internationale, Lyon, France, Aug. 23-26, 2007

“Endogenous and exogenous nitric oxide protect against intracoronary thrombosis and reocclusion after thrombolysis,” by Sheng-Kun Yao, Circulation. 1995; 92:1005-1010

“Effects of chronic baroreceptor stimulation on the autonomic cardiovascular regulation in patients with drug-resistant arterial hypertension,” by Wustmann, Hypertension 2009; 54; 530-536

### SUMMARY OF EMBODIMENTS

For some applications of the invention a subject suffering from congestive heart failure, diastolic heart failure, hypertension, and/or another condition is identified. The subject is treated by implanting an electrode on the subject's vagus nerve at a vagal site that is between (a) the vagal bifurcation with the thoracic cardiac branch, and (b) the thoracic vagal branching into the esophageal plexus. Alternatively or additionally, an electrode is implanted on the subject's aorta, at an aortic site that is between the bifurcations of the aorta with the first and fifth intercostal arteries. The subject is treated by driving a current into the electrode implantation site. The effects of driving the current into the implantation site typically include ventricular and aortic pressure reduction, an increase in aortic compliance, a decrease in sympathetic tone, and/or an increase in parasympathetic tone. These effects are typically advantageous in treating heart failure.

For some applications of the present invention, a sensing electrode is implanted in the vicinity of a non-coronary blood vessel of a subject, for example, in the vicinity of an artery, such as the subject's aorta. The sensing electrode detects an electrical parameter of the blood vessel (e.g., the aorta), and a control unit receives the detected parameter and generates an output in response to the detected parameter.

For some applications, the electrode is implanted at a site that is between 20 mm and 50 mm downstream from an aortic valve of the subject.

The electrical parameter that the sensing electrode detects is typically indicative of the subject's cardiac cycle. Thus, for some applications, cardiac-cycle-derivation functionality of the control unit detects the subject's cardiac cycle, and/or a timing parameter of the subject's blood pressure by analyzing the detected parameter. Typically, treatment functionality of the control unit generates an output, responsively to the detected parameter. For example, the treatment functionality may generate an electrical stimulus (e.g., to stimulate a blood vessel of the subject) in response to the detected parameter. Or, the treatment functionality may generate a mechanical stimulus (e.g., a pressure change at the subject's aorta for causing counterpulsation, or afterload reduction), responsively to the detected parameter. For some applications, the treatment functionality generates the mechanical stimulus using a pressure applicator, such as an intra-aortic balloon.

For some applications, the sensing electrode is placed at a first location in the vicinity of a non-coronary blood vessel of the subject, and the control unit generates an output that has an effect at (or in the vicinity of) the first location. For example, the sensing electrode may be placed on an artery that supplies the subject's penis, such as the internal pudendal artery. In response to the detected parameter, the control unit drives an electrode (e.g., the sensing electrode or a different electrode) to drive a current into the internal pudendal artery. Alternatively or additionally, the sensing electrode is placed at a first location in the vicinity of a first non-coronary blood vessel of the subject, and the control unit generates an output that has an effect at a second location within the subject's body (e.g., a location in the vicinity of a second non-coronary blood vessel). For example, the sensing electrode may be placed on the subject's aorta, and in response to the detected parameter, the control unit drives an electrode to drive a current into the subject's internal pudendal artery.

For some applications, the control unit drives a current into the aorta in response to the detected parameter. For some applications, the control unit drives the current in coordination with the subject's cardiac cycle. For example, the subject's cardiac cycle may be determined by analyzing the detected parameter, as described hereinabove. Alternatively, the cardiac cycle is detected using an ECG, and/or by taking impedance measurements, for example, using the Cheetah Reliant, described hereinabove and/or similar technology. For example, in response to detecting systole of the subject, the control unit may dilate the aorta by increasing nitric oxide (NO) secretion by the wall of the aorta by driving the current. Alternatively or additionally, in response to detecting diastole of the subject, the control unit enhances constriction of the aorta by driving the current.

For some applications of the present invention, two or more electrodes are implanted in a vicinity of an aorta of a subject. A control unit peristaltically pumps blood through the aorta by sequentially dilating portions of the aorta by facilitating nitric oxide production by the aorta by driving a current into the aorta via the electrodes. For some applications, the control unit peristaltically pumps blood through a different blood vessel of the subject, in the aforementioned manner. For example, the control unit may peristaltically pump blood through any artery, such as a renal artery or a carotid artery, or through a vein of the subject.

For some applications, the control unit receives an indication of the subject's cardiac cycle (e.g., using techniques described herein), and drives the current in coordination with the subject's cardiac cycle. Typically, the control unit peristaltically pumps blood through the aorta during systole of the subject. For some applications, during diastole of the subject, the control unit does not peristaltically pump blood through the aorta, and/or the control unit enhances constriction of the aorta by driving a diastolic current into the aorta via the electrodes.

There is therefore provided, in accordance with some applications of the present invention, a method, including:

identifying a subject as suffering from a condition selected from the group consisting of congestive heart failure, diastolic heart failure, and hypertension; and

in response to the identifying:
placing an electrode on a vagus nerve of the subject at a vagal site that is between (a) a vagal bifurcation with a thoracic cardiac branch of the subject, and (b) thoracic vagal branching into the esophageal plexus of the subject; and
treating the subject by driving a current into the vagal site, via the electrode.

For some applications, placing the electrode at the vagal site includes placing the electrode on a portion of the vagus nerve that is adjacent to a portion of an aorta of the subject that is between first and fifth intercostal arteries of the subject.

For some applications, treating the subject includes reducing ventricular pressure of the subject.

For some applications, treating the subject includes reducing aortic pressure of the subject.

For some applications, treating the subject includes reducing sympathetic tone of the subject.

For some applications, treating the subject includes increasing parasympathetic tone of the subject.

For some applications, treating the subject includes increasing aortic compliance of the subject.

For some applications, the method further includes, in response to the identifying:

placing an electrode on an aorta of the subject at an aortic site that is between first and fifth intercostal arteries of the subject; and

treating the subject by driving a current into the aortic site, via the electrode.

For some applications, treating the subject includes increasing parasympathetic tone of the subject and reducing sympathetic tone of the subject.

For some applications, treating the subject includes reducing a ratio of a low frequency component to a high frequency component of heart rate variability of the subject.

For some applications, the low frequency component is less than 0.05 Hz, and the high frequency component is between 0.15 and 0.35 Hz.

For some applications, treating the subject includes reducing a ratio of a low frequency component to a high frequency component of heart rate variability of the subject.

For some applications, the low frequency component is less than 0.05 Hz, and the high frequency component is between 0.15 and 0.35 Hz.

There is further provided, in accordance with some applications of the present invention, a method, including:

identifying a subject as suffering from a condition selected from the group consisting of congestive heart failure, diastolic heart failure, and hypertension; and

in response to the identifying:
placing an electrode on an aorta of the subject at an aortic site that is between first and fifth intercostal arteries of the subject; and
treating the subject by driving a current into the aortic site, via the electrode.

For some applications, placing the electrode at the aortic site includes placing the electrode on a portion of the aorta that is adjacent to a portion of a vagus nerve of the subject that is between (a) a vagal bifurcation with a thoracic cardiac branch of the subject, and (b) thoracic vagal branching into the esophageal plexus of the subject.

For some applications, treating the subject includes reducing ventricular pressure of the subject.

For some applications, treating the subject includes reducing aortic pressure of the subject.

For some applications, treating the subject includes reducing sympathetic tone of the subject.

For some applications, treating the subject includes increasing parasympathetic tone of the subject.

For some applications, treating the subject includes increasing aortic compliance of the subject.

For some applications, placing the electrode on the aorta includes assessing a response of the subject to placement of the electrode at a plurality of sites, and implanting the electrode at the aortic site in response to the assessing.

For some applications, treating the subject includes increasing parasympathetic tone of the subject and reducing sympathetic tone of the subject.

For some applications, treating the subject includes reducing a ratio of a low frequency component to a high frequency component of heart rate variability of the subject.

For some applications, the low frequency component is less than 0.05 Hz, and the high frequency component is between 0.15 and 0.35 Hz.

For some applications, treating the subject includes reducing a ratio of a low frequency component to a high frequency component of heart rate variability of the subject.

For some applications, the low frequency component is less than 0.05 Hz, and the high frequency component is between 0.15 and 0.35 Hz.

There is further provided, in accordance with some applications of the present invention, a method for use with one or more non-coronary blood vessels of a subject, and a body of a subject, including:

at a first location in a vicinity of one of the blood vessels, detecting an electric signal that is indicative of electrical activity at the first location due to a cardiac cycle of the subject; and

responsively thereto, generating an output at a location selected from the group consisting of: the first location, and a second location within the subject's body that is different from the first location.

For some applications, the selected location includes the first location, and generating the output includes generating the output at the first location.

For some applications, the selected location includes the second location, and generating the output includes generating the output at the second location.

For some applications, generating the output includes applying pressure to the selected location.

For some applications, the selected location includes an aorta of the subject, and applying the pressure includes counterpulsating the aorta by applying the pressure.

For some applications, the selected location includes an aorta of the subject, and applying the pressure includes reducing afterload of the subject by applying the pressure.

For some applications, generating the output includes driving a current into the selected location.

For some applications, the method further includes identifying the subject as suffering from erectile dysfunction, the selected location includes an artery of the subject that supplies a penis of the subject, and applying the electrical stimulation to the selected location includes, responsively to identifying the subject as suffering from the erectile dysfunction, treating the erectile dysfunction of the subject.

For some applications, detecting the signal includes detecting the signal at an aorta of the subject.

For some applications, detecting the signal includes detecting the signal at the artery that supplies the penis.

There is additionally provided, in accordance with some applications of the present invention, apparatus for use with one or more non-coronary blood vessels of a subject, and a body of a subject, including:

an electrode configured to be placed at a first location in a vicinity of one of the blood vessels, and to detect an electrical signal of the blood vessel;

cardiac-cycle-derivation functionality configured to derive from the signal a current phase of a cardiac cycle of the subject; and

treatment functionality configured, responsively to the derived phase, to generate an output at a location selected from the group consisting of: the first location, and a second location within the subject's body that is different from the first location.

For some applications, the apparatus further includes a pressure-applicator, and the treatment functionality is configured to generate the output by causing the pressure applicator to apply pressure to the selected location.

The present invention will be more fully understood from the following detailed description of embodiments thereof, taken together with the drawings, in which:

### BRIEF DESCRIPTION OF THE DRAWINGS

FIGS. 1A-B are schematic illustrations of electrode implantation sites, in accordance with some applications of the present invention;

FIG. 2 is a schematic illustration of an experimental setup of an experiment conducted in accordance with some applications of the present invention;

FIG. 3 is a set of graphs showing the results of stimulating a subject's vagus nerve on several physiological parameters of the subject, as determined in the experiment conducted in accordance with some applications of the present invention;

FIG. 4 is a graph showing a composite result of stimulating the subject's vagus nerve, as determined in the experiment conducted in accordance with some applications of the present invention;

FIG. 5 is a graph showing the dynamic response of a subject to the stimulation of the subject's vagus nerve, as determined in the experiment conducted in accordance with some applications of the present invention;

FIG. 6 is a graph showing the effect of stimulating an aortic site of a pig on blood pressure variability of the pig, in accordance with some applications of the present invention;

FIG. 7 is a graph showing the effect of stimulating an aortic site of a pig on heart rate variability of the pig, in accordance with some applications of the present invention;

FIGS. 8 and 9 are schematic illustrations of electrode configurations that are used, in accordance with some applications of the present invention;

FIG. 10 is a schematic illustration of an electrode implanted in a non-cardiac site in a vicinity of a subject's aorta, in accordance with some applications of the present invention;

FIGS. 11A-C are schematic illustrations of peristaltic dilation of the aorta, in accordance with some applications of the present invention;

FIG. 12 is a schematic illustration of a control unit configured to generate an output in response to a detected aortic electrical parameter, in accordance with some applications of the present invention;

FIGS. 13A-B are schematic illustrations of electrodes disposed on a self-expansible stent, in accordance with some applications of the present invention;

FIGS. 14A-B are schematic illustrations of respective views of a configuration of the self-expansible stent, in accordance with another application of the present invention;

FIGS. 15A-B are schematic illustrations of respective views of an alternative configuration of the self-expansible stent, in accordance with some applications of the present invention;

FIGS. 16A-B are schematic illustrations of respective views of a further alternative configuration of the self-expansible stent, in accordance with some applications of the present invention;

FIG. 17 is a plot of an aortic voltage signal recorded in an aorta of a pig, in an experiment conducted in accordance with some applications of the present invention;

FIG. 18 is a plot showing frequency components of the aortic voltage signal of FIG. 17, as extracted from the raw aortic voltage signal in accordance with some applications of the present invention;

FIG. 19 is a plot comparing a frequency component of the aortic voltage signal of FIG. 17 to the pig's ECG and blood pressure signals, in accordance with some applications of the present invention; and

FIG. 20 is a graph showing blood pressure changes measured in five experiments conducted on four pigs, in accordance with some applications of the present invention.

### DETAILED DESCRIPTION OF EMBODIMENTS

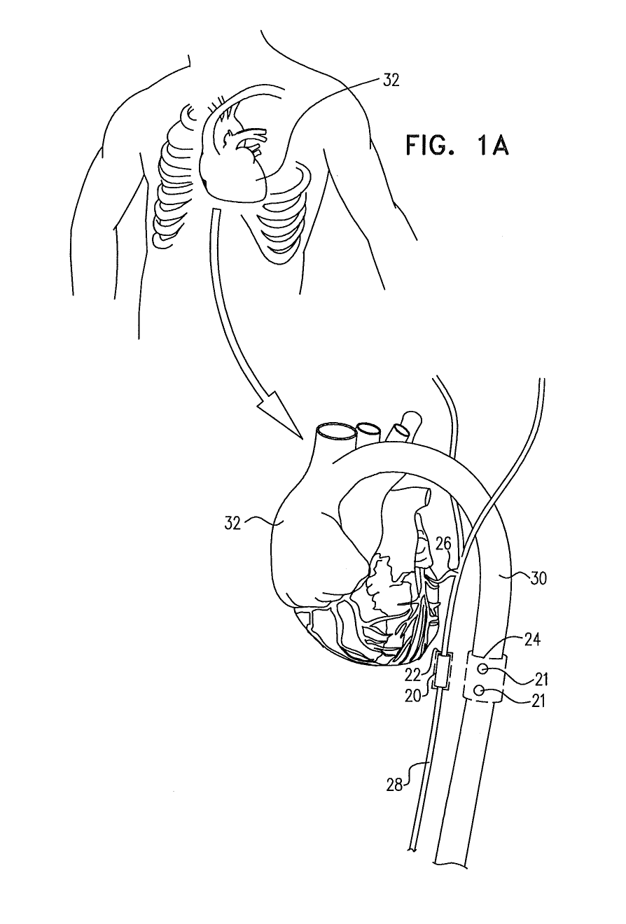

Reference is now made to FIGS. 1A-B, which are schematic illustrations of a vagal site 22 and an aortic site 24 of a subject, in accordance with some applications of the present invention. For some applications, at least one vagal electrode 20 and at least one aortic electrode 21 are implanted, respectively, at vagal site 22 and aortic site 24. In FIG. 1A, vagus nerve 28 is shown separated from aorta 30 for illustrative purposes, although typically the vagus nerve is disposed adjacently to the aorta at aortic site 24, as shown in FIG. 1B.

For some applications of the invention, a subject suffering from congestive heart failure, diastolic heart failure, and/or hypertension is identified. The subject is treated by implanting an electrode on the subject's vagus nerve at vagal site 22 that is between (a) vagal bifurcation 26 with thoracic cardiac branch, and (b) the thoracic vagal branching into the esophageal plexus. Alternatively or additionally, one or more aortic electrodes 21 are implanted on the subject's aorta 30, at aortic site 24 that is between the bifurcations of the descending thoracic aorta with the first and fifth intercostal arteries 27 and 29. For some applications, aortic electrode 21 is implanted on a portion of the aorta that is adjacent to vagal site 22. For some applications, vagal electrode 20 is implanted on a portion of the vagus nerve that is adjacent to aortic site 24. The subject is treated by driving a current into one or more of the electrode implantation sites. The effects of driving the current into the implantation site typically include ventricular and aortic pressure reduction, an increase in aortic compliance, a decrease in sympathetic tone, and/or an increase in parasympathetic tone.

Typically, the lowering of the subject's blood pressure is achieved by driving the current into one or both of the implantation sites, without causing a substantial change in the subject's heart rate. For some applications, there is no substantial effect on the heart rate, because the current is driven into a site that is further from the CNS than the thoracic cardiac bifurcation 26, and therefore does not have a substantial effect on nerves that directly innervate the subject's heart 32. (For some applications, stimulating the vagus nerve distally to bifurcation 26 also has a heart rate lowering effect, but it is hypothesized by the inventors that this effect is mediated through central controls rather than direct efferent stimulation of the heart.) Typically, the lowering of the subject's blood pressure is achieved due to physiological responses that are in addition to any effects on the firing rate of the subject's baroreceptors, due to the applied current. Further typically, vagal electrode 20 and/or aortic electrodes 21 stimulate at least non-baroreceptor vagal terminals of vagal nerve 28.

For some applications, aortic electrodes 21 are disposed inside the aorta (i.e., electrodes 21 are intravascular electrodes). Alternatively or additionally, the electrodes are disposed in a wall of the aorta. Further alternatively or additionally, vagal electrode 20 is a cuff-electrode (or a different design) that is placed around, or in contact with, the vagus nerve. For some applications, electrode 20 and/or electrodes 21 are chronically implanted at sites 22 and/or 24.

For some applications, the current is driven into the electrode implantation site in coordination with the subject's cardiac cycle and/or respiratory cycle. Alternatively, the current is driven independently of the subject's cardiac cycle and/or respiratory cycle.

For some applications, driving current into aortic site 24, via electrodes 21, dilates the aorta by increasing nitric oxide (NO) secretion by the wall of the aorta, and/or by increasing the secretion of another vasodilation mediator from the wall of the aorta. For some applications, driving current into aortic site 24, via electrodes 21, dilates the aorta by stimulating efferent nerve ending. For some applications, driving current into aortic site 24, via electrodes 21, dilates the aorta by direct electrical hyperpolarization of the vascular smooth muscle. For some applications, the current has a frequency of between 5 Hz and 50 Hz. For some applications, the current has an amplitude of between 1 mA and 15 mA, e.g., between 2 mA and 3 mA. For some applications, a current having two pulses to eight pulses, e.g., three pulses to five pulses, per cardiac cycle, is driven into the aorta to dilate the aorta. In accordance with respective applications, the current is delivered continuously or intermittently. The current may thus be applied, for example: (a) as an endless train of pulses, (b) during scheduled non-contiguous daily stimulation periods, or (c) during each of at least 24 consecutive hours.

For some applications, driving current into vagal site 22, via electrode 20 stimulates parasympathetic nerve endings and elicits a parasympathetic response. For some applications, driving the current into the vagal site stimulates sympathetic nerve endings, and inhibits sympathetic signaling. For some applications, driving current into aortic site 24, via electrodes 21, has a similar effect on the vagus nerve (i.e., a vagal response), due to the proximity of aortic site 24 to vagal site 22, and/or due to vagal nerve endings that are located at the aortic site. For some applications, driving current into the aortic site generates an aortic response, as described hereinabove, in addition to generating the aforementioned vagal response.

For some applications, vagal site 22 is mechanically stimulated, for example, by mechanically stimulating the vagus nerve at the vagal site, and/or by mechanically stimulating aortic site 24, such that the vagal site also becomes stimulated. For some applications, the vagal site is stimulated using piezoelectric actuator terminals, an electrical motor, and/or an electroactive polymer actuator. For some applications, a balloon is placed in the vicinity of the vagal site, and is actuated to mechanically stimulate the vagus nerve using an external pump.

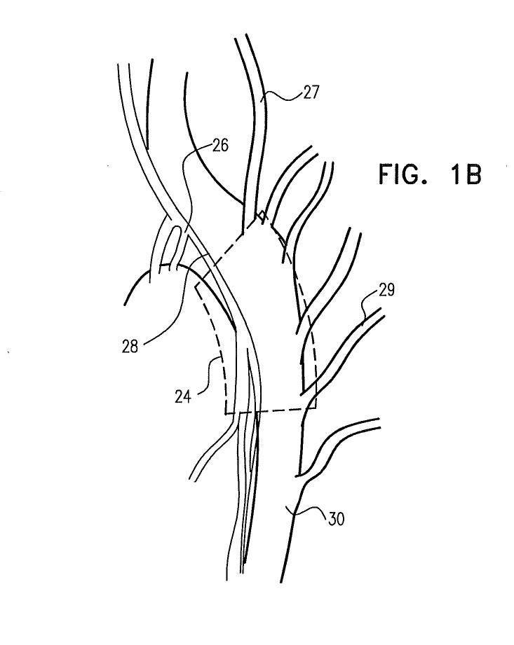

Reference is now made to FIG. 2, which is a schematic illustration of an experimental setup of an experiment conducted in accordance with some applications of the present invention. Cuff electrodes were placed around a pig's vagus nerve at the following four locations:
(1) cervical location 40;
(2) proximal thoracic location 42 which is proximal to (i.e., closer to the CNS than) where the vagus has crossed the aorta;
(3) medial thoracic location 44, 1-2 cm below the aortic arch as the vagus nerve runs alongside the descending aorta, and just distal to (i.e., further from the CNS than) the thoracic cardiac branch bifurcation with the vagus nerve; and
(4) distal thoracic location 46, just distal to (i.e., in a downstream direction along the aorta from) the crossing of the azygos vein with the aorta, and approximately 3 cm distal to (i.e., further from the CNS than) the thoracic cardiac branch bifurcation with the vagus nerve.

Reference electrodes e1 and e2 were placed inside the pig's body, as shown in FIG. 2. Three Millar pressure transducers M1, M2, and M3 were placed, respectively, in the left ventricle, the proximal descending aorta and in the abdominal aorta proximal to the iliac bifurcation. A Transonic flow transducer 41 was positioned around the aortic root. Three minutes of continuous electrical stimulation was applied to each of the sites. Respective sites of the pig's vagus were stimulated in accordance with the parameters provided in Table 1.

TABLE 1
Stimulation parameters

| Active | Ref. | amplitude | freq | pulse | stimulation |
|---|---|---|---|---|---|
| pole | pole | [mA] | [Hz] | width | duration |
| 46 Distal | e1 | 5 | 50 | 1-1 ms * | 3 min |
| 44 Medial | e1 | 5 | 50 | 1-1 ms | 3 min |
| 42 Proximal | e1 | 5 | 50 | 1-1 ms | 3 min |
| 40 Cervical | e2 | 5 | 50 | 1-1 ms | 3 min |

* i.e., a 1 ms positive pulse, followed by a 1 ms symmetric negative pulse

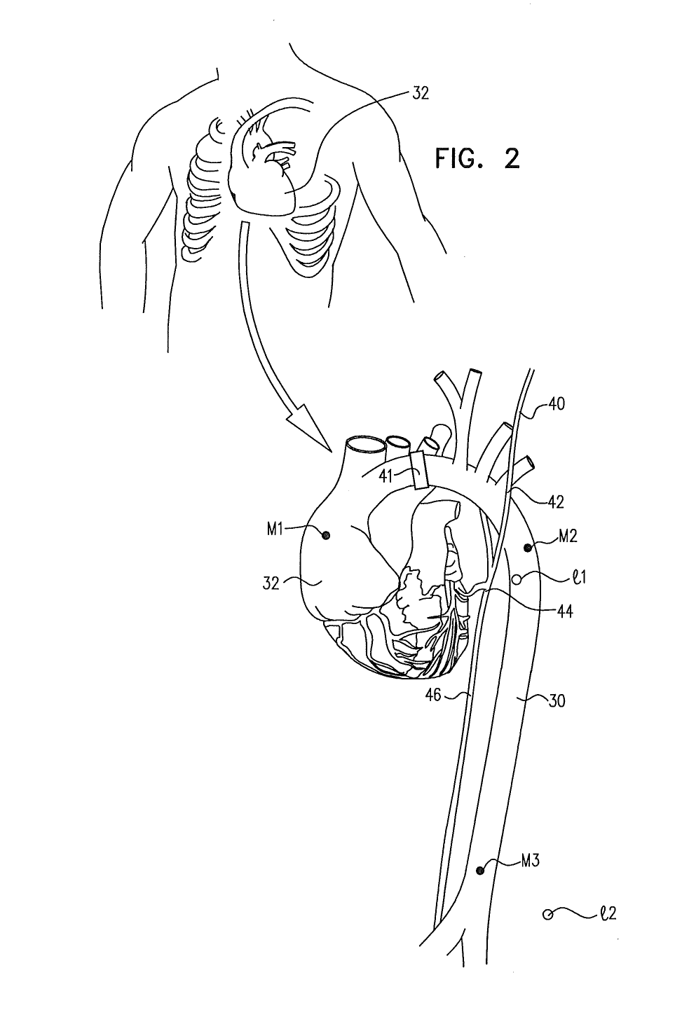

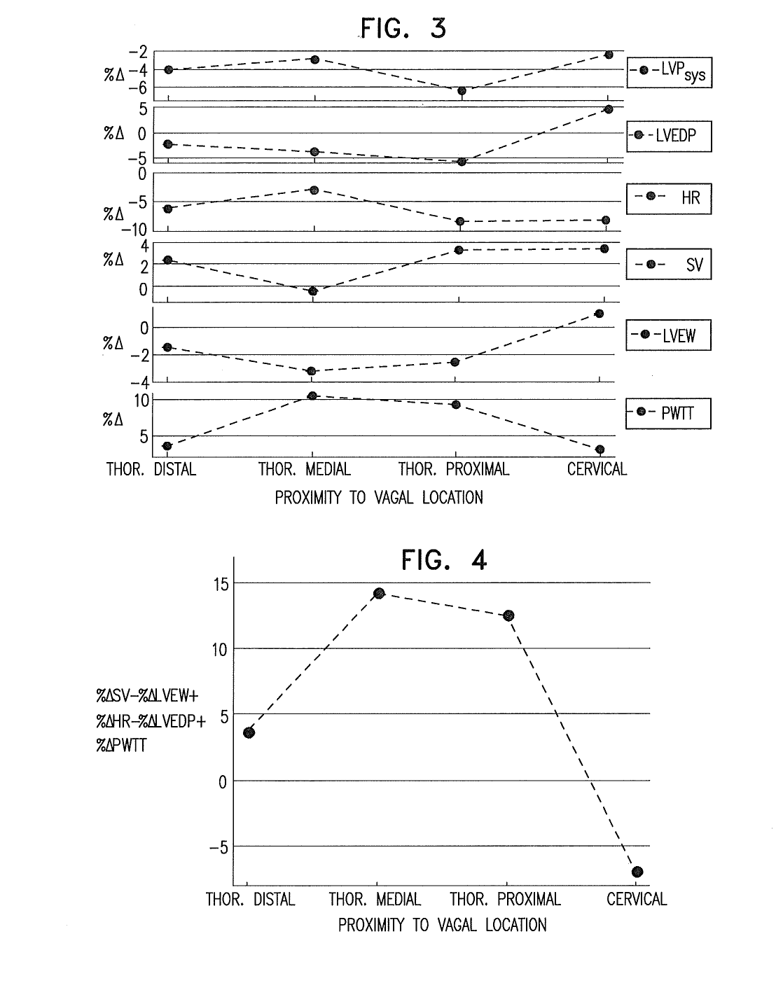

Reference is now made to FIG. 3, which is a set of graphs showing the results of stimulating the pig's vagus on several physiological parameters of pig, as determined in the experiment described with reference to FIG. 2. The following parameters were determined.
LVPsys—Average systolic left ventricular pressure during the ejection phase (aortic valve opening to aortic valve closure).
LVEDP—Left ventricular end diastolic pressure.
HR—Heart rate.
SV—Stroke volume as measured in the aortic root.
LVEW—Left ventricular external work. The integral of the product of left ventricular pressure and aortic flow during ejection phase.
PWTT—Pulse wave travel time between two measuring points along the aorta. PWTT is correlated to the square root of the diameter of the aorta divided by stiffness. Hence, increased PWTT (decreased pulse wave velocity) is associated with decreased aortic wall tonus.

The numeric values shown in the graphs of FIG. 3 represent the average of each parameter, for respective stimulation sites, during the entire stimulation regime. The following observations can be made regarding the graphs shown in FIG. 3:
Electrical stimulation at all locations induced a reduction of average systolic left ventricular pressure during the ejection phase and heart rate. The systolic left ventricular pressure reduction was maximal in the proximal site and minimal in the cervical site.
The left ventricular end diastolic pressure was reduced in the thoracic sites and increased in the cervical site.
Heart rate reduction was maximal in the proximal thoracic and cervical sites.
Stroke volume did not exhibit a clear trend, as the medial thoracic site yielded a slight decrease and the other sites resulted in 2-4% increase.
Left ventricular external work, which is related to cardiac consumption, was lower as a result of stimulation of the thoracic sites and higher while stimulating the cervical site.
Stimulation at all of the sites resulted in an increase in pulse wave travel time (i.e., a decrease in aortic tonus). Stimulation of the proximal and medial sites resulted in the largest pulse delay along the aorta.

Reference is now made to FIG. 4, which is a graph showing a composite result of stimulating the pig's vagus, as determined in this experiment. In order to evaluate each of the stimulation sites with one parameter, a first order scoring function was applied. The percentage change in each of the parameters shown in the graph of FIG. 3 was added to the total score, and its sign was determined according to the presumed beneficial direction. Left ventricular external work and left ventricular end diastolic pressure, which are targeted to be reduced (when treating patients suffering from hypertension, for example), were added with negative signs. Pulse wave travel time and stroke volume were added with positive signs. The heart rate reduction was also assigned a positive score.

The function results are plotted in the graph shown in FIG. 4. It may be observed that the thoracic medial site has the highest score, and all of the thoracic sites achieved positive scores. The cervical vagal site achieved an overall negative score, since, although it had a positive effect on heart rate (i.e., heart rate reduction), its effect on pressure and work reduction was non-beneficial across the entire stimulation regime.

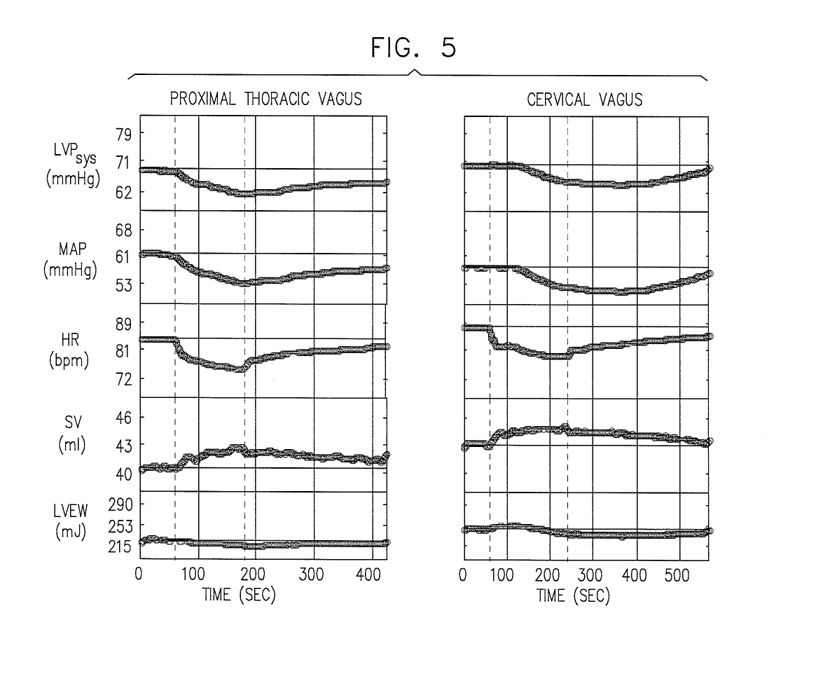

Reference is now made to FIG. 5, which is a graph showing the dynamic response of the pig to the stimulation of the pig's vagus nerve, as determined in this experiment. The dynamic response to stimulation of the proximal thoracic and the cervical sites is shown in FIG. 5. The beginnings and ends of the stimulation period are marked with dashed vertical lines, at approximately 60 sec and 180 sec on the proximal thoracic vagus graph, and 60 sec and 230 sec on the cervical vagus graph. Heart rate response in both cases was immediate and continued for the duration of the stimulation period. Similarly, there was stroke volume elevation for the duration of the stimulation, due to stimulation at both sites. The pressure and left ventricular external work responses were not similar, however. The proximal thoracic site generated almost immediate pressure and work reduction. In the cervical site, the pressure reduction appeared only late in stimulation (possibly, as a secondary indirect phenomenon), and the left ventricular external work parameter responded with initial increases that were present across most of the stimulation regime.

In view of the results presented herein, it is hypothesized, by the inventors of the present application that, as compared to stimulation of the cervical vagus, stimulation of thoracic vagal sites, as described herein, results in (a) a greater overall desired response with respect to ventricular and aortic blood pressure reduction and decreased aortic tonus, and (b) a more rapid response time to the stimulation. The inventors further hypothesize that placing electrodes on an aortic site that is between the first and the fifth intercostal arteries of a human subject, will generate a similar response to the response of the pig to the placement of electrodes at the proximal, medial and distal sites, in the experiment described herein. The inventors additionally hypothesize that placing electrodes on a vagal site that is adjacent to the aforementioned aortic site will also generate a similar response.

The inventors further hypothesize that stimulating a subject's vagus nerve and/or aorta at the sites specified herein is beneficial for treating the subject, such that the subject's (a) ventricular blood pressure, aortic blood pressure, and/or aortic tonus is reduced, without causing (b) a substantial reduction in the subject's heart rate. Conversely, stimulating a subject's vagus nerve and/or aorta at a more proximal site (i.e., at a site along the vagus nerve that is closer to the CNS, and/or at a site along the aorta that is further upstream) may cause a greater reduction in the subject's heart rate and a smaller reduction in the subject's ventricular blood pressure, aortic blood pressure, and/or aortic tonus.

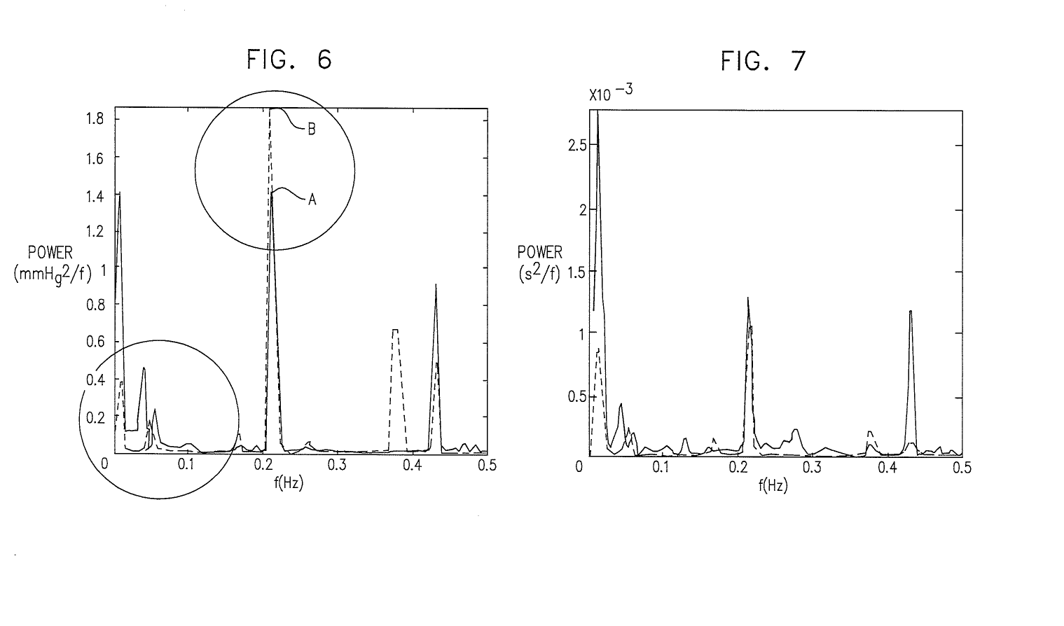

Reference is now made to FIG. 6, which is a graph showing the effect of stimulating an aortic site of a pig on blood pressure variability of the pig, in accordance with some applications of the present invention. Two aortic electrodes were placed inside the pig's aorta at an aortic site as described hereinabove, i.e., between the bifurcations of the descending thoracic aorta with the first and fifth intercostal arteries. To generate the graph shown in FIG. 6, the baseline blood pressure variability of the pig was measured (while the electrodes were not applying current to the aortic site). The baseline blood pressure variability is denoted in the graph by the solid curve. The curve was generated by collecting the baseline data for seven minutes. Subsequently, the electrodes were driven to drive a current into the aortic site having the following parameters: amplitude 10 mA, frequency 125 Hz, and pulses that were 2 ms on, 2 ms off. Blood pressure was measured during the stimulation period. The dotted curve in FIG. 6 shows the blood pressure variability based on seven minutes of the measured blood pressure during the stimulation period.

It may be observed that the effect of the stimulation on the blood pressure variability was to decrease the low frequency components of the blood pressure (those less than 0.15 Hz, e.g., less than 0.05 Hz) and to increase the high frequency components (those in the range of 0.15-0.35 Hz). For example, the frequency component at about 0.21 Hz increases from peak A to peak B, as shown. Thus, the stimulation at the aortic site caused a decrease in the ratio of low frequency components to the high frequency components (“the LF:HF ratio”). In accordance with the Laitinen article cited hereinabove in the Background, which is incorporated herein by reference, a decrease in the LF:HF ratio is indicative of inhibition of sympathetic activity and/or an increase of parasympathetic activity. This is because the low frequency components of the blood pressure variability are indicative of sympathetic activity, and the high frequency components are indicative of parasympathetic vagal activity. This experiment, therefore, not only shows a decrease in the LF:HF ratio, but also, inhibition of sympathetic activity and increase of parasympathetic activity.

Reference is now made to FIG. 7, which is a graph showing the effect of stimulating an aortic site of a pig on heart rate variability of the pig, in accordance with some applications of the present invention. It is noted that the pig used to generate the results shown in FIG. 7 was a different pig from the pig used to generate the results shown in FIG. 6.

Two aortic electrodes were placed inside the pig's aorta at an aortic site as described hereinabove, i.e., between the bifurcations of the descending thoracic aorta with the first and fifth intercostal arteries. To generate the graph shown in FIG. 7, the baseline heart rate variability of the pig was measured, while the electrodes were not applying current to the aortic site. The baseline heart variability is denoted in the graph by the solid curve. The curve was generated by collecting the baseline data for seven minutes. The electrodes were driven to drive a current into the aortic site having the following parameters: amplitude 10 mA, frequency 125 Hz, and pulses that were 2 ms on, 2 ms off. The heart rate of the pig was measured during the stimulation period. The dotted curve in FIG. 7 shows the heart rate variability based on seven minutes of the measured blood pressure during the stimulation period.

It may be observed that the effect of the stimulation on the heart rate variability was to decrease the low frequency components of the blood pressure (those less than 0.15 Hz, e.g., less than 0.5 Hz). Thus, the stimulation at the aortic site caused a decrease in the ratio of low frequency components to the high frequency components (“the LF:HF ratio”). In accordance with the Wustmann article cited hereinabove in the Background, which is incorporated herein by reference, a decrease in the LF:HF ratio of heart rate variability is indicative of inhibition of sympathetic activity and/or an increase of parasympathetic vagal activity. In this experiment, an increase in parasympathetic activity is seen.

It is noted that although it may be observed in FIG. 7 that stimulation of the pig caused a decrease in frequency components of the heart rate variability above 0.4 Hz, such components are not indicative of parasympathetic activity. Only the high frequency components up to around 0.35 Hz indicate parasympathetic activity, in accordance with the article, “Heart rate variability,” Eur Heart J, Vol. 17, March 1996, and, in particular, FIG. 4 thereof. Although some frequency components in the range of 0.15-0.35 Hz were decreased (e.g., at about 0.23 Hz), the decrease in these frequency components was small relative to the decrease in the low frequency components (e.g., at about 0.02 Hz). Therefore, the overall effect of the stimulation was to cause a decrease in the LF:HF ratio.

In accordance with the results shown in FIGS. 6 and 7, for some applications, a subject suffering from congestive heart failure, diastolic heart failure, and/or hypertension is treated by placing electrodes at an aortic site and/or a vagal site, as described herein. Parasympathetic activity of the subject is increased and/or sympathetic activity of the subject is decreased by driving a current into the site.

Reference is now made to FIG. 8, which is a schematic illustration of an electrode configuration that is used for stimulating an aortic site of a subject, in accordance with some applications of the present invention. For some applications, one or more aortic electrodes 21 are disposed on a loop 50. The loop is transvascularly placed inside the subject's aorta at the aortic site, such that the aortic electrodes contact the intravascular surface of the aorta at the aortic site. When the electrodes have been placed in contact with the intravascular surface of the aorta, current is driven into the aorta via the electrodes, in accordance with the methods described hereinabove. For example, the LASSO 2515 Variable Circular Mapping Catheter, manufactured by Biosense Webster, may be used for loop 50.

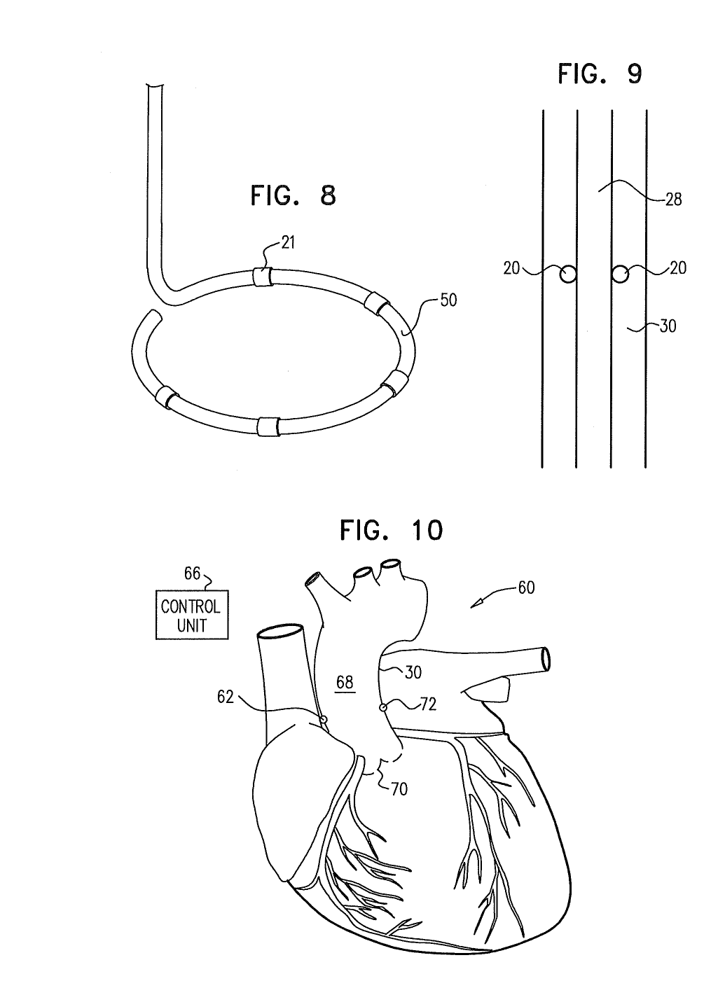

Reference is now made to FIG. 9, which is a schematic illustration of an electrode configuration that is used for stimulating vagal and/or aortic sites of a subject, in accordance with some applications of the present invention. For some applications, two electrodes 20 are placed on respective sides of vagus nerve 28, such that the electrodes are in contact with both the vagus nerve and the aorta.

For some applications, a subject is anesthetized for the purpose of performing a cardiac intervention and/or a different intervention on the subject. While the subject is in an anesthetized state, it is desirable that the subject has reduced afterload and increased preload relative to the subject's normal levels of afterload and preload. Thus, for some applications, while a subject is in an anesthetized state, the methods described herein are applied to the subject in order to reduce afterload and/or to increase preload of the subject.

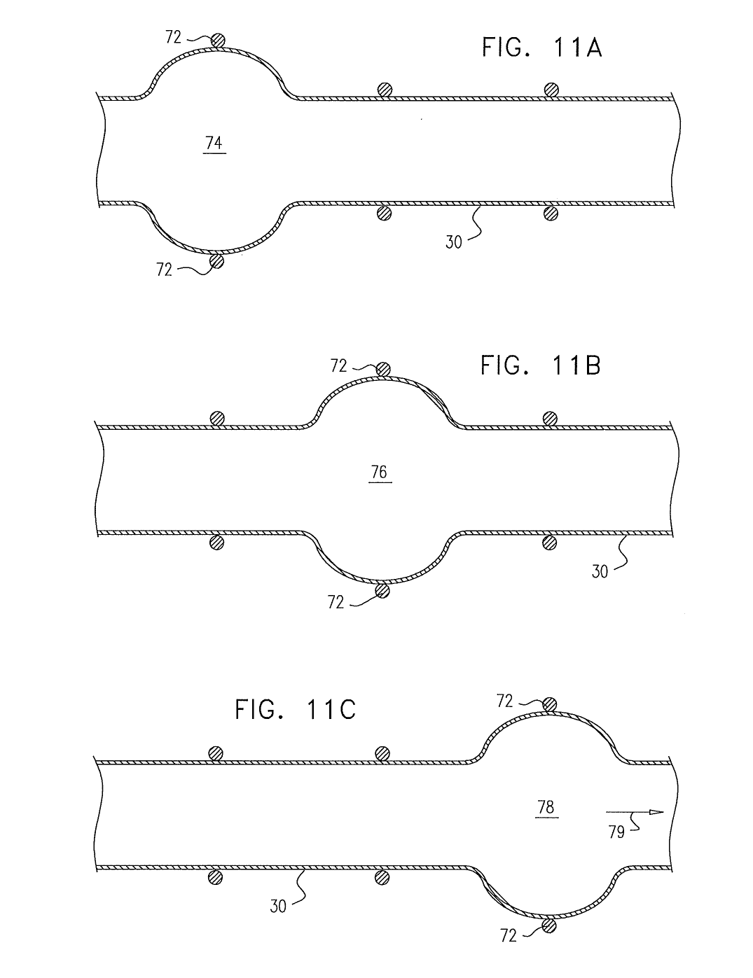

Reference is now made to FIG. 10, which is a schematic illustration of apparatus 60, comprising at least one electrode 62 implanted at a non-cardiac site in a vicinity of a subject's aorta 30, in accordance with some applications of the present invention. For some applications, electrode 62 detects an electrical parameter of the subject's aorta, and a control unit 66 receives the detected parameter and generates an output in response to the detected parameter.

For some applications, control unit 66 is disposed inside the subject's body, e.g., in a vicinity of the subject's aorta 30, or remote therefrom, similar to the implanted pulse generator of a standard cardiac pacemaker. Alternatively, the control unit is disposed outside the subject's body.

For some applications, electrode 62 is disposed inside the aorta. Alternatively or additionally, the electrode is disposed in a non-cardiac site in a vicinity of the aorta, and/or in a wall of the aorta. For some applications, electrode 62 is chronically implanted at the site in the vicinity of the aorta. Alternatively the electrode is implanted temporarily, for example, for a period of four weeks or less. For some applications, at least two electrodes 62 are implanted in the subject. One of the electrodes is placed inside the aorta, and another of the electrodes is placed outside the aorta. For some applications, first and second electrodes 62 are placed within the aorta at a longitudinal distance from each other of between 10 mm and 30 mm and/or at a radial distance from each other of less than 10 degrees. For some applications, one or more electrodes 62 are placed in the subject's ascending aorta and one or more electrodes are placed in the subject's aortic arch and/or descending aorta. For some applications, ten or more electrodes, for example 20 electrodes are implanted inside the aorta. Typically, electrode 62 is implanted in a site of the ascending aorta 68 at a site that is between 20 to 50 mm downstream from an aortic valve 70 of the subject.

For some applications, control unit 66 detects the subject's cardiac cycle, and/or a timing parameter of the subject's blood pressure by analyzing the detected parameter. For some applications, the control unit drives a current into the aorta, or into a different blood vessel, in response to the detected parameter. Examples of such detecting and current application are described hereinbelow.

For some applications, the control unit drives the current in coordination with the subject's cardiac cycle. Alternatively, control unit 66 drives a current into the subject's aorta, or a different blood vessel, independently of the subject's cardiac cycle.

For some applications, the control unit drives the current into the aorta via sensing electrode 62. Alternatively or additionally, apparatus 60 comprises one or more additional driving electrodes 72, and the control unit drives the current into the aorta via the driving electrodes. Typically, the placement parameters of the driving electrodes are similar to those described hereinabove, with respect to sensing electrode(s) 62. For some applications, the driving electrodes are oriented to have a surface area of between 3 square mm and 15 square mm, e.g. between 5 square mm and 12 square mm, in contact with tissue of the aorta.

For some applications, control unit 66, by driving a current into the aorta, dilates the aorta by increasing nitric oxide (NO) secretion by the wall of the aorta. Typically, the control unit dilates the aorta in response to detecting an indication of systole of the subject. For some applications, dilating the aorta during systole reduces the left ventricular afterload of the subject, and thereby increases the subject's stroke volume and/or ejection fraction. Alternatively, the aorta may be dilated during systole for a different purpose.

For some applications, the control unit dilates the aorta by configuring the current to have a frequency of between 5 Hz and 20 Hz, e.g., between 10 Hz and 15 Hz. For some applications, the current has an amplitude of between 1 mA and 5 mA, e.g., between 2 mA and 3 mA. For some applications, a current having two pulses to eight pulses, e.g., three pulses to five pulses, per cardiac cycle, is driven into the aorta to dilate the aorta.

For some applications, control unit 66 enhances constriction of the aorta by driving a current into the aorta. For example, the control unit may enhance constriction of the aorta in response to the control unit detecting an indication of diastole of the subject. For some applications, enhancing constriction of the aorta during diastole elevates diastolic blood pressure, thereby increasing coronary perfusion, and/or the supply of blood to organs of the subject's body other than the heart. Alternatively, constriction of the aorta may be enhanced during diastole for a different purpose.

For some applications, the control unit enhances constriction of the aorta by driving a current having a frequency of between 40 Hz and 70 Hz. For some applications, the current has an amplitude of between 5 mA and 20 mA, e.g., between 8 mA and 15 mA. For some applications, a current having ten pulses to twenty pulses, e.g., thirteen pulses to seventeen pulses, per cardiac cycle, is driven into the aorta to enhance constriction of the aorta.

For some applications, control unit 66, (a) in response to detecting systole of the subject, dilates the aorta by increasing nitric oxide (NO) secretion by the wall of the aorta by driving a current into the aorta, and (b) in response to detecting diastole of the subject, enhances constriction of the aorta by driving a current into the aorta. For example, the control unit may dilate the aorta during every systole, and enhance constriction of the aorta during intermittent diastoles. Alternatively, the control unit may dilate the aorta during intermittent systoles, and enhance constriction of the aorta during every diastole. Further alternatively, the control unit may dilate the aorta during every systole, and enhance constriction of the aorta during every diastole. Typically, a suitable protocol is selected based on the medical condition of the subject.

For some applications, a sensing electrode 62 is implanted in the vicinity of a non-coronary blood vessel of the subject that is not the aorta. Alternatively or additionally, as described hereinabove, sensing electrode 62 is implanted in the vicinity of the aorta. The sensing electrode detects an electrical parameter of the blood vessel (e.g., the aorta), and a control unit receives the detected parameter and generates an output in response to the detected parameter. The electrical parameter that the sensing electrode detects is typically indicative of the subject's cardiac cycle. Thus, for some applications, cardiac-cycle-derivation functionality of the control unit derives the subject's cardiac cycle, and/or a timing parameter of the subject's blood pressure by analyzing the detected parameter.

Typically, treatment functionality of the control unit generates an output, responsively to the detected parameter. For example, the treatment functionality may generate an electrical stimulus (e.g., to stimulate a blood vessel of the subject, as described herein) in response to the detected parameter. Or, the treatment functionality may generate a mechanical stimulus (e.g., a pressure change at the subject's aorta for causing counterpulsation, or afterload reduction), responsively to the detected parameter.

For some applications, sensing electrode 62 is placed at a first location in the vicinity of a non-coronary blood vessel of the subject, and the control unit generates an output that has an effect at (or in the vicinity of) the first location. For example, as described hereinabove, the control unit may electrically stimulate the aorta, responsively to sensing at the aorta. Alternatively, the sensing electrode may be placed on an artery that supplies the subject's penis, such as the internal pudendal artery. In response to the detected parameter, the control unit drives an electrode (e.g., the sensing electrode or a different electrode) to drive a current into the internal pudendal artery. Alternatively or additionally, the sensing electrode is placed at a first location in the vicinity of a first non-coronary blood vessel of the subject, and the control unit generates an output that has an effect at a second location within the subject's body (e.g., a location in the vicinity of a second non-coronary blood vessel). For example, the sensing electrode may be placed on the subject's aorta as described hereinabove, and, in response to the detected parameter, the control unit drives an electrode to drive a current into the subject's internal pudendal artery.

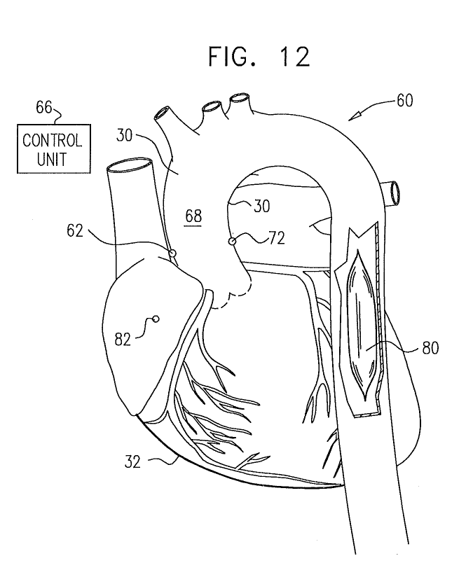

Reference is now made to FIGS. 11A-C, which are schematic illustrations of peristaltic dilation of aorta 30, in accordance with some applications of the present invention. For some applications of the invention, control unit 66 (FIG. 10) peristaltically pumps blood through the aorta by sequentially dilating portions of the aorta by facilitating nitric oxide production by the aorta by driving a current into the aorta via two or more electrodes disposed at respective longitudinal positions on the aorta. Typically, during diastole, control unit 66 causes a region 74 of the aorta to dilate by driving a current through the most-downstream electrodes of electrodes 72 (as shown in FIG. 11A). The current is sequentially driven through the remaining electrodes, causing regions 76 and 78 to dilate (as shown in FIGS. 11B and 11C respectively), and causing blood to flow in an upstream direction, in the direction of arrow 79, to enhance coronary artery perfusion. Alternatively, peristalsis generated as described is used to generate downstream-directed enhanced blood flow.

Although applications are described herein according to which the aorta is peristaltically pumped, the scope of the present application includes applying to any blood vessel in the subject's body, the methods and apparatus for peristaltic pumping that are described herein. For example, the scope of the present invention includes peristaltically pumping blood through a subject's renal artery, carotid artery, or a vein, by peristaltically dilating the blood vessel.

Typically, the parameters of the current for dilating the aorta are as described hereinabove. Typically, the parameters of the electrodes (i.e., the number and spacing of the electrodes) are as described hereinabove. Further typically, the electrodes are configured to induce dilation with a spacing in time of 10 ms to 50 ms. For some applications, the electrodes are disposed longitudinally along the aorta with a longitudinal spacing therebetween of 150%-250% of the local diameter of the aorta and/or of 1-5 cm. The spacing may be maintained, for example, by a housing to which the electrodes are coupled (e.g., a flexible stent) or by sutures or adhesives which couple the electrodes to the aorta. As appropriate for the level of peristaltic flow desired, the time for a peristaltic wave to be generated and to travel from the most downstream to the most upstream electrode (or in the opposite direction) typically ranges from 0.25 second to about 2 seconds.

For some applications, control unit 66 receives an indication of the subject's cardiac cycle (e.g., via sensing electrode 62, which may be placed at the aorta or at a different non-coronary blood vessel, as described hereinabove), and peristaltically pumps blood in the aorta by driving the current in coordination with the subject's cardiac cycle. For some applications, the control unit peristaltically pumps blood through the aorta during systole of the subject. For some applications, a peristaltic wave of constriction of the aorta is generated as well as the peristaltic wave of dilation described hereinabove. The peristaltic wave of constriction is behind the peristaltic wave of dilation, and pushes the blood in the peristaltic wave of dilation. For example, while region 76 of the aorta is dilated (as shown in FIG. 11B), region 74 is constricted (constriction not shown), and subsequently, while region 78 is dilated, region 76 is constricted.

For some applications, during diastole of the subject, control unit 66 (a) does not peristaltically pump blood through the aorta, and/or (b) enhances constriction of the aorta by driving a diastolic current into the aorta via the electrodes. Typically, the parameters of the diastolic current for enhancing constriction of the aorta are as described hereinabove.

For some applications, control unit 66 peristaltically pumps blood proximally during diastole by generating a proximally-directed peristaltic wave of dilation and/or contraction using the techniques described hereinabove.

For some applications, control unit 66 peristaltically dilates the aorta during intermittent or all systoles, and/or enhances constriction of the aorta during intermittent or all diastoles.

Typically, control unit 66 comprises a battery. Alternatively, the control unit is powered wirelessly, e.g., by being irradiated with electromagnetic radiation, and/or ultrasound radiation from outside the subject's body, or by extracting energy from the subject's body. For example, the control unit may be disposed inside the subject's aorta, and configured to extract energy from the flow of blood through the aorta. Alternatively or additionally, the control unit may extract energy from the subject's gastric acid.

Reference is now made to FIG. 12, which is a schematic illustration of apparatus 60, in accordance with some applications of the present invention. For such applications, a pressure applicator, such as an intra-aortic balloon pump 80 is implanted in a subject's aorta. Control unit 66 pumps the intra-aortic balloon pump in response to the electrical parameter of the aorta or of a different non-coronary blood vessel that is detected by electrode 62.

For some applications, in addition to, or instead of pump 80, apparatus 60 includes at least one cardiac electrode 82 implanted in a vicinity of the subject's heart 32. Control unit 66 drives a current into the subject's heart, via the cardiac electrode, in response to the electrical parameter of the aorta, or of a different non-coronary blood vessel that is detected by sensing electrode 62. For some applications, the control unit defibrillates or cardioverts the subject's heart by driving the current into the subject's heart, in response to aortic sensing, other non-coronary blood vessel sensing, and/or in response to sensing on the heart.

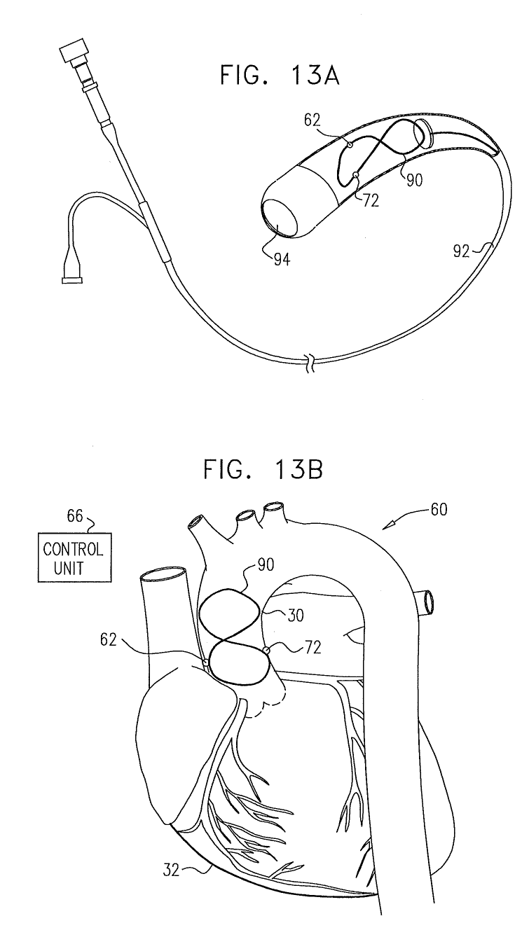

Reference is now made to FIGS. 13A-B, which are schematic illustrations of electrodes 62 and/or 72 disposed on a self-expansible stent 90, in accordance with some applications of the present invention. Typically, the stent is inserted into the subject's aorta 30, via a catheter 92. The stent is in a contracted state when it is inside the catheter, and expands automatically inside the aorta upon exiting the distal end 94 of catheter.

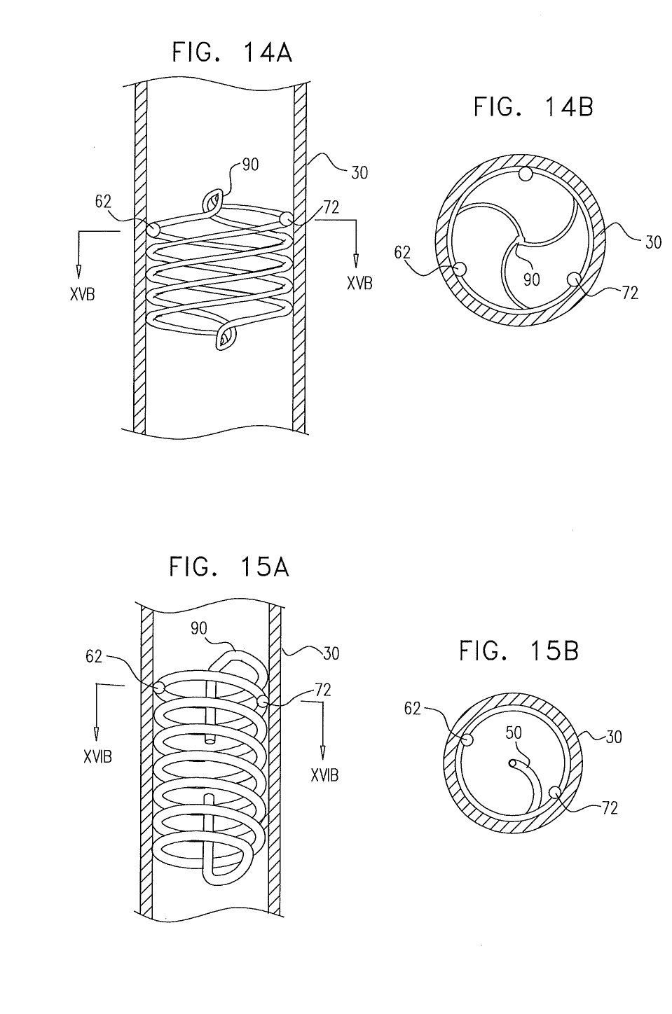

Reference is now made to FIGS. 14A-B, which are schematic illustrations of respective views of a configuration of self-expansible stent 90, in accordance with some applications of the present invention. For some applications, stent 90 (as shown) is shaped as two or more spirals. The spirals are in contracted states inside catheter 92, and are held in place inside aorta 30 by expanding inside aorta 30.

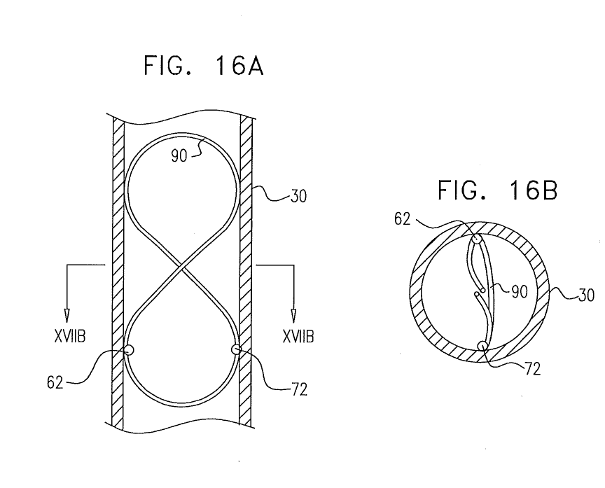

Reference is now made to FIGS. 15A-B, which are schematic illustrations of respective views of an alternative configuration of self-expansible stent 90, in accordance with some applications of the present invention. For some applications, stent 90 (as shown) is shaped as a coil. The coil is in a contracted state inside catheter 92, and is held in place inside aorta 30 by expanding inside aorta 30.

Reference is now made to FIGS. 16A-B, which are schematic illustrations of respective views of a further alternative configuration of self-expansible stent 90, in accordance with some applications of the present invention. For some applications, stent 90 (as shown) is shaped as a figure-of-eight. The figure-of-eight is in a contracted state inside catheter 92, and is held in place inside aorta 30 by expanding inside aorta 30.

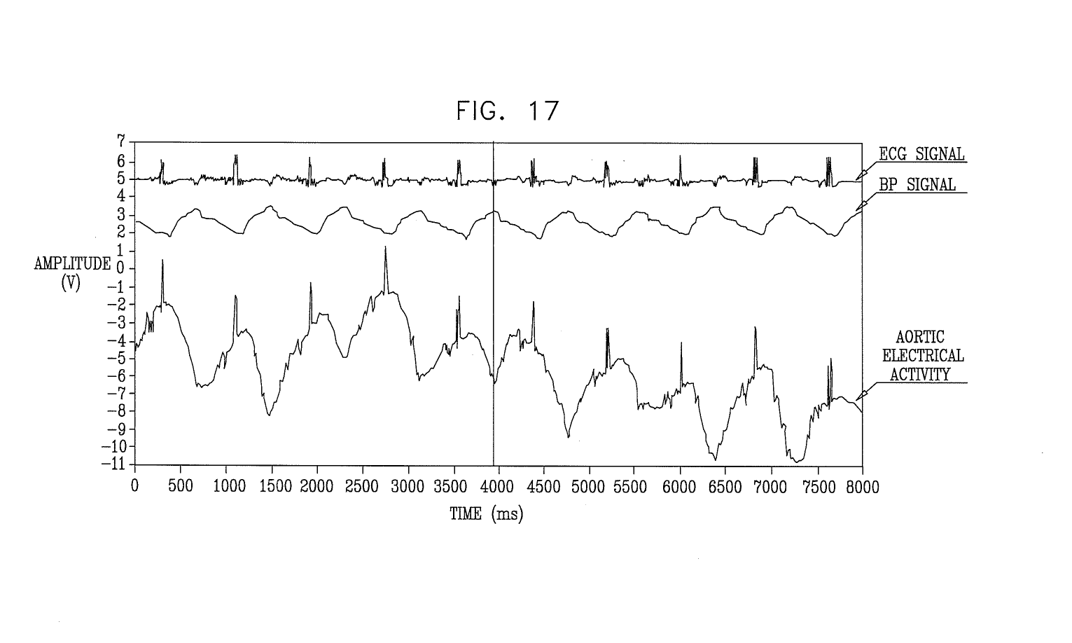

Reference is now made to FIG. 17, which is a graph of aortic electrical activity recorded in an aorta of a pig, in an experiment conducted in accordance with some applications of the present invention. Ten electrodes were placed in an aorta of a pig close to the aortic valve, and the voltage within the aorta was recorded via four of the ten electrodes. The graph shows the variation of the voltage within the aorta plotted against time. In addition, and concurrently, the pig's ECG and blood pressure were measured. The graph additionally shows the concurrent ECG and blood pressure measurements, which were respectively recorded with an external ECG electrode and with an intra-aortic blood pressure sensor.

Based upon the data in FIG. 17 and in other experiments carried out by the inventors, the inventors have identified relationships between the cardiac cycle and the voltage recorded in the aorta. For example:

(1) There is a sharp peak in the aortic voltage about 50-100 ms before the onset of the aortic pressure rise due to systole. For example, at 2000 ms there is an onset of the pressure rise, and about 70 ms before this onset there is a sharp peak in the aortic voltage.

(2) Shortly before the onset of the aortic pressure decline due to diastole, the aortic voltage reaches a minimum. For example, there is a solid vertical line through the graph at about 3950 ms, at which point, the aortic voltage is at a local minimum. At about 4000 ms, diastole begins.

(3) A signal component in the measured aortic voltage corresponds to, and appears quite similar to, the R-wave recorded with an external ECG electrode, shown in the top trace. For example, the spike in the aortic voltage signal at 6000 ms corresponds to the R-wave in the ECG signal at 6000 ms.

Thus, the inventors have found that important mechanical events (onset of aortic pressure rise and aortic pressure decline) and electrical events (the R-wave) can be identified by aortic sensing, and, for some applications, are processed and used to trigger a medical device, such as an intra-aortic balloon pump or a pulse generator. The inventors hypothesize that mechanical and electrical events of other blood vessels that are indicative of the subject's cardiac cycle can be detected by sensing electrical activity of the other blood vessels. For example, a sensing electrode placed in the vicinity of a non-coronary artery that is not the aorta (e.g., the internal pudendal artery) may be used to detect electrical and/or mechanical events of the artery that are indicative of a phase of the subject's cardiac cycle.

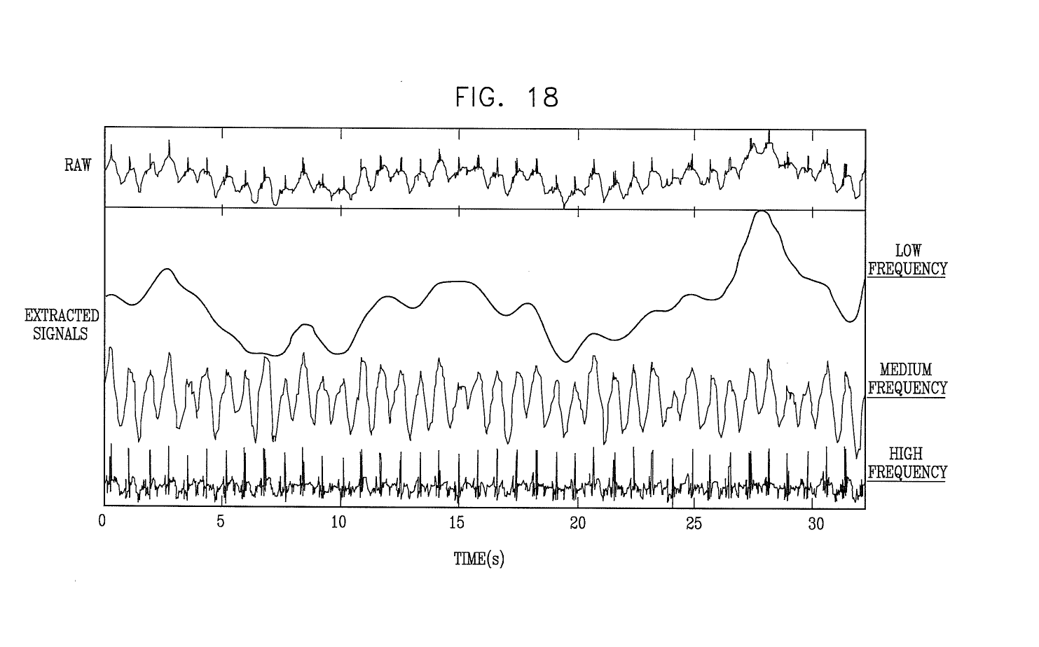

Reference is now made to FIG. 18, which is a plot showing frequency components extracted from the raw aortic voltage signal of FIG. 17, in accordance with some applications of the present invention. The aortic voltage signal was separated into three frequency components, a low-frequency component, a medium-frequency component, and a high-frequency component.

Reference is now made to FIG. 19, which shows the high frequency component of the aortic voltage signal plotted together with an ECG recorded by the external electrode and the recorded blood pressure. It was observed by the inventors that the high frequency component has a similar pattern to the ECG signal, as can be seen in FIG. 19. Furthermore, there is a relationship between the occurrence of systole and diastole (which are indicated by the dots on the blood pressure plot), and the plot of the high frequency signal. As such, for some applications of the invention, an ECG signal of a subject is detected by sensing an electrical parameter in the subject's aorta. For some applications, a subject's ECG signal is detected by sensing electrical activity of another non-coronary blood vessel of the subject. For example, a sensing electrode placed in the vicinity of a non-coronary artery other than the aorta (e.g., the internal pudendal artery) may be used to detect the subject's ECG signal.

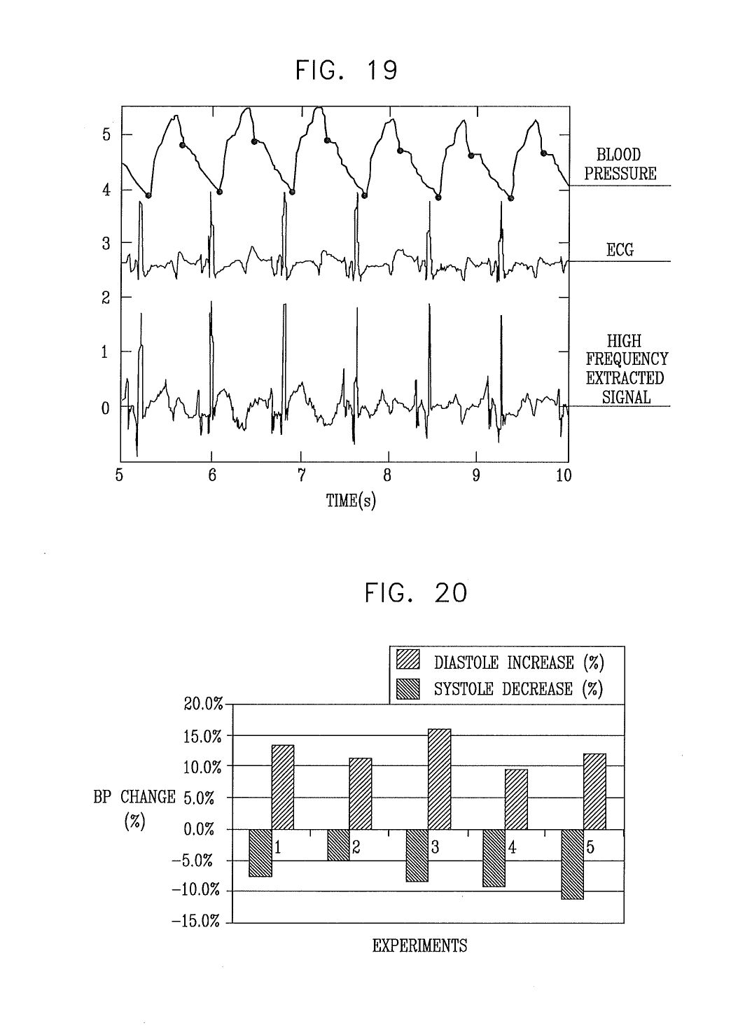

Reference is now made to FIG. 20, which is a graph showing blood pressure changes measured in five experiments conducted on four pigs, in accordance with some applications of the present invention. In each experiment, the pig was opened surgically, and electrodes (having configurations described hereinbelow) were implanted on the aortic wall. In each of the five experiments, currents having respective parameters were driven into the pigs' aortas during systole and diastole. The systolic currents dilated the aorta (thus decreasing systolic aortic blood pressure), by increasing nitric oxide (NO) secretion by the wall of the aorta. The diastolic currents enhanced constriction of the aorta (thus increasing diastolic aortic blood pressure).

The parameters of the electrodes used, and the currents with which the aortas were stimulated in each of the five experiments, were in accordance with Table 2 below. In Table 2, “Type 1” electrodes denotes Pathfinder electrodes manufactured by Cardima (CA) [product no. 01-161003]. “Type 2” electrodes denotes electrodes, which were custom made for the inventors, each of the custom-made electrodes having a length of 13.3 mm to 13.5 mm, having a diameter of 0.52 mm, and being pointed at a distal end thereof. The custom-made electrodes were oriented to have approximately 10 sq mm of surface area in contact with the wall of the aorta and to be at a minimum distance of 10 mm from each other. All of the electrodes were implanted in the ascending aortas of the pigs.

TABLE 2

| | | Systolic | Diastolic |
|---|---|---|---|
| Experiment | Electrode | current | current |
| 1 | Type 1 | 2 mA | 8 mA monophasic |
| | | monophasic | |
| | | 20 Hz | 40 Hz |
| | | 6 pulses per | 15 pulses per |
| | | cardiac cycle | cardiac cycle |
| 2 | Type 2 | 5 mA | 12 mA monophasic |
| | | monophasic | |
| | | 30 Hz | 80 Hz |
| | | 2 pulses per | 15 pulses per |
| | | cardiac cycle | cardiac cycle |
| 3 | Type 2 | 2 mA | 8 mA monophasic |
| | | monophasic | |
| | | 20 Hz | 50 Hz |
| | | 6 pulses per | 15 pulses per |
| | | cardiac cycle | cardiac cycle |
| 4 | Type 2 | 2 mA | 7 mA monophasic |
| (This | | monophasic | |
| experiment | | 12 Hz | 50 Hz |
| was | | 4 pulses per | 16 pulses per |
| performed | | cardiac cycle | cardiac cycle |
| on the | | | |
| same pig | | | |
| as that of | | | |
| experiment | | | |
| 3) | | | |
| 5 | Type 1 | 1 mA | 10 mA |
| | | monophasic | monophasic |
| | | 20 Hz | 60 Hz |
| | | 6 pulses per | 4 pulses per |
| | | cardiac cycle | cardiac cycle |

The mean decrease in the systolic blood pressure, as a result of the systolic currents, was 8.3±2.3% (mean±standard deviation). The mean increase in diastolic blood pressure, as a result of the diastolic currents, was 12.4±2.5% (mean±standard deviation).

For some applications, the techniques described herein are practiced in combination with techniques described in PCT Publication WO 07/013,065 to Gross, which is incorporated herein by reference. For some applications, the techniques described herein are practiced in combination with the techniques described in PCT application WO 09/095,918, entitled “Peristaltic pump for treatment of erectile dysfunction,” to Gross, which claims priority from US Patent Application 2009/0198097 to Gross, the PCT application and the US application being incorporated herein by reference. For some applications, the techniques described herein are practiced in combination with the techniques described in US Patent Application 2009/0198097 to Gross.

For some applications, the methods described herein are performed in combination with the techniques described in PCT Application WO 09/095,920 to Gross, which is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method, comprising:
identifying a subject as suffering from a condition selected from the group consisting of congestive heart failure, diastolic heart failure, and hypertension; and
in response to the identifying:
placing an electrode on a vagus nerve of the subject at a vagal site that is between (a) a vagal bifurcation with a thoracic cardiac branch of the subject, and (b) thoracic vagal branching into the esophageal plexus of the subject; and
treating the subject by driving a current into the vagal site, via the electrode.

2. The method according to claim 1, wherein placing the electrode at the vagal site comprises placing the electrode on a portion of the vagus nerve that is adjacent to a portion of an aorta of the subject that is between first and fifth intercostal arteries of the subject.

3. The method according to claim 1, wherein treating the subject comprises reducing ventricular pressure of the subject.

4. The method according to claim 1, wherein treating the subject comprises reducing aortic pressure of the subject.

5. The method according to claim 1, wherein treating the subject comprises reducing sympathetic tone of the subject.

6. The method according to claim 1, wherein treating the subject comprises increasing parasympathetic tone of the subject.

7. The method according to claim 1, wherein treating the subject comprises increasing aortic compliance of the subject.

8. The method according to claim 1, further comprising, in response to the identifying:
placing an electrode on an aorta of the subject at an aortic site that is between first and fifth intercostal arteries of the subject; and
treating the subject by driving a current into the aortic site, via the electrode.

9. (canceled)

10. The method according to claim 1, wherein treating the subject comprises reducing a ratio of a low frequency component to a high frequency component of heart rate variability of the subject.

11. The method according to claim 10, wherein the low frequency component is less than 0.05 Hz, and wherein the high frequency component is between 0.15 and 0.35 Hz.

12. The method according to claim 1, wherein treating the subject comprises reducing a ratio of a low frequency component to a high frequency component of blood pressure variability of the subject.

13. The method according to claim 12, wherein the low frequency component is less than 0.05 Hz, and wherein the high frequency component is between 0.15 and 0.35 Hz.

14. A method, comprising:
identifying a subject as suffering from a condition selected from the group consisting of congestive heart failure, diastolic heart failure, and hypertension; and
in response to the identifying:
placing an electrode on an aorta of the subject at an aortic site that is between first and fifth intercostal arteries of the subject; and
treating the subject by driving a current into the aortic site, via the electrode.

15. (canceled)

16. The method according to claim 14, wherein treating the subject comprises reducing ventricular pressure of the subject.

17. The method according to claim 14, wherein treating the subject comprises reducing aortic pressure of the subject.

18. The method according to claim 14, wherein treating the subject comprises reducing sympathetic tone of the subject.

19. The method according to claim 14, wherein treating the subject comprises increasing parasympathetic tone of the subject.

20. (canceled)

21. The method according to claim 14, wherein treating the subject comprises increasing aortic compliance of the subject.

22. (canceled)

23. The method according to claim 14, wherein treating the subject comprises reducing a ratio of a low frequency component to a high frequency component of heart rate variability of the subject.

24. The method according to claim 23, wherein the low frequency component is less than 0.05 Hz, and wherein the high frequency component is between 0.15 and 0.35 Hz.

25. The method according to claim 14, wherein treating the subject comprises reducing a ratio of a low frequency component to a high frequency component of heart rate variability of the subject.

26. The method according to claim 25, wherein the low frequency component is less than 0.05 Hz, and wherein the high frequency component is between 0.15 and 0.35 Hz.

27. A method for use with one or more non-coronary blood vessels of a subject, and a body of a subject, comprising:
at a first location in a vicinity of one of the blood vessels, detecting an electric signal that is indicative of electrical activity at the first location due to a cardiac cycle of the subject; and
responsively thereto, generating an output at a location selected from the group consisting of: the first location, and a second location within the subject's body that is different from the first location.

28-29. (canceled)

30. The method according to claim 27, wherein generating the output comprises applying pressure to the selected location.

31. The method according to claim 30, wherein the selected location includes an aorta of the subject, and wherein applying the pressure comprises counterpulsating the aorta by applying the pressure.

32. The method according to claim 30, wherein the selected location includes an aorta of the subject, and wherein applying the pressure comprises reducing afterload of the subject by applying the pressure.

33. The method according to claim 27, wherein generating the output comprises driving a current into the selected location.

34. The method according to claim 33, further comprising identifying the subject as suffering from erectile dysfunction, wherein the selected location includes an artery of the subject that supplies a penis of the subject, and wherein applying the electrical stimulation to the selected location comprises, responsively to identifying the subject as suffering from the erectile dysfunction, treating the erectile dysfunction of the subject.

35. The method according to claim 34, wherein detecting the signal comprises detecting the signal at an aorta of the subject.

36. The method according to claim 34, wherein detecting the signal comprises detecting the signal at the artery that supplies the penis.

37-46. (canceled)
